(19) United States
(12) Patent Application Publication
(10) Pub. No.: US 2020/0175255 A1
(43) Pub. Date: Jun. 4, 2020

(54) DEVICE FOR DETERMINING FEATURES OF A PERSON

(71) Applicant: Bayer Business Services GMBH, Köln (DE)

(72) Inventors: Ali KÜCÜKCAYIR, Köln (DE); Jürgen HOHMANN, Odenthal (DE)

(73) Assignee: Bayer Business Services GMBH, Köln (DE)

(21) Appl. No.: 16/341,615

(22) PCT Filed: Oct. 13, 2017
(86) PCT No.: PCT/EP2017/076177

§ 371 Date: Apr. 12, 2019

(30) Foreign Application Priority Data
Oct. 20, 2016 (EP) 16194848.4

(51) Int. Cl. G06K 9/00; A61B 5/024; G06Q 30/02; G10L 25/63; G01J 5/00; G06F 3/0482

(52) U.S. Cl. CPC G06K 9/00221; A61B 5/024; G06Q 30/0271; G06K 2009/00322; G01J 5/0025; G06F 3/0482; G10L 25/63

(57) ABSTRACT

The invention relates to the determination of features of a person. The invention relates to a device and to a method for determining features of a person in order to be able to better engage with the person in an interaction with the person.

The present invention relates to determining features of a person. The subjects of the present invention are a device and a method for determining features of a person to be able to better engage with the person in an interaction with the person.

In particular in modern cities, there are numerous situations in everyday life in which two people who do not know each other interact with one another. One typical example is a purchase situation. In this case, a customer having a desire or a need approaches a salesperson; the salesperson consults with the customer and sells the customer a product, which fulfills the wish or satisfies the need. The interaction takes place according to typical principles which apply similarly to a variety of consultancy situations.

There are numerous cases in which it would be advantageous if the consultant knew more about his customer. The more the consultant knows about the customer, the better he can engage with the individual concerns of the customer. A customer cannot always clearly articulate his needs. It is also conceivable that a customer is in a negative state. It is conceivable that the consultancy situation is unpleasant to the customer; however, it is also conceivable that he is mentally and/or physically stressed and brings this stress into the situation. The rapid pace of modern life and the chronic lack of time resulting therefrom also have a negative effect on such a situation.

If the consultant were to recognize such stress, he could thus engage better with the person. He could thus, for example, adapt his gestures, mode of expression, and/or volume. He could adapt the surroundings of the conversation by light, sounds, odors, temperature, air movement, and the like. The fact that an empathetic consultation has a high level of relevance was proven in the context of a study with patients having colds in the pharmacy: a reduction of the duration of the cold by 1 day and an alleviation of the symptoms by 16% was able to be achieved by more empathic consultation by the pharmacist (source: Rakel D P et al. Practitioner Empathy and the Duration of the Common Cold Fam Med 2009; 41(7):494-501.).

A demand therefore exists for determining the physical and/or mental state of a person. In this case, it is not only important to analyze the state of the counterpart for sales situations. For example, official business is unpleasant to many persons and it would be favorable to know whether a counterpart is tense and which circumstances have a calming effect on him.

However, it is important to be able to recognize not only negative states but rather also positive ones. Humans in a positive state are frequently more open and, for example, more ready to take part in a survey on the street.

The determination of the physical and/or mental state of a person is to be carried out as much as possible without action of the person, so as not to provide additional hurdles or distract from the conversation. The determination is not to influence the person. It is therefore to take place in a contactless manner.

The contactless registration of physical features plays a role in various fields: video cameras having facial recognition are used in access control systems; infrared cameras are used in airports to be able to recognize travelers having a febrile infection.

There are also devices, using which multiple parameters can be registered simultaneously.

US 2016/0191822A1 discloses, for example, a device for facial recognition which is additionally equipped with heart rate detection. The device is used to be able to differentiate the faces of real persons from faces on photographs.

EP1522256A1 discloses a device for recording items of information by means of various sensors. A bio-information sensor is used to register items of information, for example, the heart rate, the galvanic skin response (GSR), and the skin temperature of a person. The bio-information sensor is worn on the body. In addition, there are sensors such as a camera and a microphone which register the surroundings of the person.

DE102006038438A1 discloses a device for determining medical and biometric data of a living being. The device comprises a first unit for authentication of the living being on the basis of a physical feature, a second unit for determining a medical function of the person or an item of information about the person, and a third unit for transmitting the determined medical and/or biometric data. The device is to be usable in health screening, in personal supervision, and in entry or access control.

In contrast, the subjects of claims 1 to 15 are not disclosed in the prior art.

A first subject matter of the present invention is a device comprising
one or more sensors for the contactless determination
   of the sex of a person and
   the association of the person with an age group and
   the skin temperature of the person and
   the heart rate of the person and
   the mood of the person
a first display screen,
means for reading out the one or more sensors,
means for processing the data read out from the one or more sensors and for associating the read-out and processed data with one or more visual representations of the data, and
means for displaying the visual representations on the first display screen.

A further subject matter is a method for analyzing a first person, comprising the following steps:
recognition of the presence of a first person,
contactless determination of the following features of the first person with the aid of one or more sensors:
   the sex and
   the association with an age group and
   the skin temperature and
   the heart rate and
   the mood
display of the physical and mental features on a first display screen.

The invention will be explained in greater detail hereafter without differentiating between the subjects of the invention (device, method). Rather, the following explanations are to apply similarly to all subjects of the invention, independently of the context (device, method) in which they are performed.

For clarification, it is to be noted that it is not the goal of the present invention to register features of persons without their knowledge. In many countries of the earth, there are provisions in data protection law and personal law which are to be observed in every case. Although the registration of features of a person takes place contactlessly according to the invention and without action of the person, the consent of the person for the registration of the features has to exist. The aspects with respect to data protection law are also to be observed in the processing of personal data, of course. Finally, the present invention is to be useful to those persons from whom the physical and/or mental features are registered. The specific embodiment of the present invention is accordingly to have means which enable a person to recognize and reject or consent to a registration of physical and/or mental features.

In a first step of the method according to the invention, the presence of a person who is positioned opposite to the device according to the invention in a suitable manner is recognized.

The device according to the invention is typically stationed at a specific location and registers immediate surroundings of the device using one or more sensors. A mobile device which can be set up as needed at a location is also conceivable. The device according to the invention is typically unmoving, however, if it is used for registering features of a person in its immediate surroundings.

Changes in these immediate surroundings are registered. The immediate surroundings relate, typically, to an angle range of 30° to 180° around the device and a distance range of 0.1 to 10 m. If there is a person in these immediate surroundings, it is recognized by the device that it is a person.

Appropriate sensors are typically used for recognizing the presence of a person, for example, image sensors, distance meters, and the like. An image sensor on which the person or parts of the person are depicted is typically used.

An image sensor is a device for recording two-dimensional images from light in an electrical manner. In most cases, semiconductor-based image sensors are used, which can record light up into the middle infrared.

Examples of image sensors in the visible range and in the near infrared are CCD sensors (CCD: charge-coupled device) and CMOS sensors (CMOS: complementary metal-oxide-semiconductor).

The image sensor is connected to a computer system on which software is installed, which decides, for example, on the basis of a feature analysis of the depiction whether the imaged content is a person or not.

It is preferably determined on the basis of the presence or absence of a human face in a depiction of the surroundings of the device according to the invention registered by the image sensor whether a person is present or absent, respectively.

For this purpose, a region is preferably registered by the image sensor in which the face of a person who stops in front of the device is typically located.

Furthermore, light from the face of the person has to be incident on the image sensor. The ambient light is typically used. If the device according to the invention is located outside, thus, for example, sunlight can be used during the day. If the device according to the invention is located in a building, artificial light which illuminates the interior of the building can be used. However, it is also conceivable to use a separate light source in order to illuminate the face of the person optimally. The wavelength range in which the light source emits light is preferably adapted to the sensitivity of the image sensor used.

It can be determined with the aid of a face location method whether a face is depicted on the image sensor. If the probability that a face is depicted on the image sensor is greater than a definable threshold value (for example, 90%), it is then assumed by the computer system that a person is present. If the probability is less than the threshold value, in contrast, it is assumed by the computer system that a person is not present.

Face location methods are presently implemented in many digital cameras.

Simple face location methods search for characteristic features in the depiction, which could originate from eyes, nose, and mouth of a person, and decide on the basis of the geometrical relationships of the features to one another whether it could be a face (two-dimensional geometrical measurement).

The use of neuronal networks or similar technologies for recognizing (locating) a face is also conceivable.

The computer system and the image sensor can be configured so that the image depicted on the image sensor is supplied to an image analysis in definable time intervals (for example, every second) in order to ascertain the probability that a face is present on the image.

However, it is also conceivable that the system is configured in such a way that an image is recorded by the image sensor and supplied to an analysis as soon as a distance sensor registers that something is located in the immediate surroundings in front of the device according to the invention.

After the presence of a person has been recognized, various features of the person are registered. The person, of whom the features are registered, will also be referred to hereafter as the “person to be analyzed” or as the “analyzed person” or as the “first person”.

The registered features are preferably displayed opposite to a further person who is not the first person. They will also be referred to hereafter as the “second person”.

The device according to the invention comprises sensors, using which physical and mental features of the first person can be determined.

The invention relates to determining the state of a person. The state of a person can be understood as the total of all physical and mental features of the person. However, all physical and mental features do not have to be registered to determine the state of a person. Even a small number of features can give information about the state of a person. It can already be determined on the basis of a small number of features whether a person has certain needs and/or wishes.

Physical features of a person are understood as bodily features of the person. Examples of physical features are height, weight, sex, and the association with an age group. These features may be “read” directly on the body of the person.

The sex of a person is registered as a physical feature according to the invention. An image sensor which is connected to a computer system is preferably used for the contactless determination of the sex.

The face of a person is preferably registered to determine the sex.

The same components are preferably used for the determination of the sex which are also used for the determination of the presence of the person.

After a face has been located in a depiction, characteristic features of the face can be analyzed to decide whether it is a man or a woman. The analysis of a face for determining physical and/or mental features is also referred to here as facial recognition (while the face location only has the task of recognizing the presence of a face).

In one preferred embodiment, an artificial neuronal network is used to determine the sex from the face recording.

Numerous approaches are described in the literature for how features such as the sex of a person can be determined from a digital depiction of the face (see, for example, Okechuwku A. Uwechue, Abhijit S. Pandya: Human Face Recognition Using Third-Order Synthetic Neural Networks, Springer Science+Budiness Media, LLC., 1997, ISBN 978-1-4613-6832-8; Stan Z. Li, Anil K. Kain (Editors), Handbook of Face Recognition, Second Edition, Springer 2011, ISBN 978-0-85729-931-4; Maria De Marsico et al.: Face Recognition in Adverse Conditions, Advances in Computational Intelligence and Robotics Book Series 2014, ISBN 978-1-4666-5966-7; Thirimachos Bourlai (Editor): Face Recognition Across the Imaging Spectrum, Springer 2016, ISBN 978-3-319-28501-6; http://www.iis.fraunhofer.de/de/ff/bsy/tech/bildanalyse/shore-gesichtsdetektion.html).

The age represents a further bodily feature. No method is hitherto known, using which the exact age of a person can be determined via a contactless sensor. However, the approximate age may be determined on the basis of various features which can be registered in a contactless manner. In particular the appearance of the skin, above all in the face, gives information about the approximate age. Since an exact age is not determinable in a contactless manner (unless one asks the person), the association with an age group is the goal in the present case.

According to the invention, the association with an age group (as with the sex of a person) is also determined by means of an image sensor which is connected to a computer system, on which facial recognition software runs. The same hardware is preferably used for determining the association with an age group as for the determination of the sex.

An artificial neuronal network is preferably used for determining the association of a person with an age group.

The age groups may be defined arbitrarily in principle in this case, for example, one could define a new age group every 10 years: persons in the age from 0 to 9, persons in the age from 10 to 19, persons in the age from 20 to 29, etc.

However, the breadth of variation in the age-specific features which can be registered in a contactless manner for humans in the age from 0 to 9 is substantially greater than that for humans in the age from 20 to 29 years. An allocation into age groups which takes the breadth of variation into consideration is thus preferable.

An age may also be estimated in years and this age may be specified together with a relative or absolute error or a probability.

Further physical features which may be contactlessly determined with the aid of an image sensor are, for example: height, weight, hair color, skin color, hair length/hair fullness, spectacles, posture, gait, inter alia.

To determine the height of the person, it is conceivable, for example, to depict the head of the standing person on the image sensor and to determine the distance of the person from the image sensor using a distance meter (for example, using a laser distance measuring device, which measures the runtime and/or the phasing of a reflected laser pulse). The height of the person then results from the location of the depicted head on the image sensor and the distance of the person from the image sensor in consideration of the optical elements between image sensor and person.

The weight of a person may also be estimated from the height and the width of the person. Height and width may be determined by means of the image sensor.

In addition to the physical features mentioned, mental features are also registered. Mental features are to be understood as features which permit inferences about the mental state of a person. In the final analysis, the mental features are also bodily features, i.e., features which can be recognized and registered on the body of a human. In contrast to the solely physical features, however, the mental features are to be attributed either directly to a mental state or they accompany a mental state.

One feature which is a direct expression of the mental state of a person is, for example, the facial expression: a smiling person is in a better mental state than a crying person or an angry person or a fearful person.

In one embodiment of the present invention, an image sensor having connected computer system and software is used for the facial recognition in order to analyze the facial expression and recognize the mood of the person (happy, sad, angry, fearful, surprised, inter alia).

The same hardware can be used to determine the facial expression which is also used to determine the age.

The following moods are preferably differentiated: angry, happy, sad, and surprised.

One feature which is an indirect expression of the mental state of a person is, for example, the body temperature. An elevated body temperature is generally a sign of an illness (with accompanying fever); and illness generally has a negative effect on the mental state; persons with fever “usually do not feel well.”

In one preferred embodiment, the temperature of the skin is preferably determined in the face, preferably on the forehead of the person.

Infrared thermography can be used for the contactless temperature measurement (see, for example, Jones, B. F.: A reappraisal of the use of infrared thermal image analysis in medicine. IEEE Trans. Med. Imaging 1998, 17, 1019-1027).

A further feature which can be an indirect expression of the mental (and physical) state of a person is the heart rate. An elevated heart rate can indicate nervousness or fear or also an organic problem.

Various methods are known, using which the heart rate can be determined contactlessly by means of an image sensor having a connected computer system.

Oxygen-rich blood is pumped into the arteries with every heartbeat. Oxygen-rich blood has a different color than oxygen-poor blood. The pulsing color change can be recorded and analyzed using a video camera. The skin is typically irradiated using red or infrared light for this purpose and the light reflected from the skin is captured by means of a corresponding image sensor. In this case, the face of a person is typically registered, since it is typically not covered by clothing. More specific details can be taken, for example, from the following publication and the references listed in the publication: http://www.cv-foundation.org/openaccess/content_cvpr_workshops_2013/W13/papers/Gault_A_Fully_Automatic_2013 CVPR_paper.pdf.

Another option is the analysis of head movements, which are caused by the pumping of blood into the head of a person (see, for example, https://people.csail.mit.edu/mrub/vidmag/papers/Balakrishnan_Detecting_Pulse_from_2013_CVPR_paper.pdf).

The head movement is preferably analyzed by means of a video camera. In addition to the movements which are caused by the pumping of blood into the head (pumping movements), the analyzed person could execute further head movements (referred to here as “natural head movements”), for example, those head movements which are executed when the analyzed person permits his gaze to wander. It is conceivable to ask the person to be analyzed to keep the head still for the analysis. However, as described at the outset, the registration according to the invention of features is to take place substantially without action of the person to be analyzed. A video sequence of the head of the person to be analyzed is therefore preferably preprocessed in order to eliminate the natural head movements. This is preferably performed in that facial features, for example, the eyes, the eyebrows, the nose and/or the mouth and/or other features are fixed in successive image recordings of the video sequence at fixed points in the image recordings. Thus, for example, if the center point of the pupils travel as a result of a rotation of the head within the video sequence from two points x^{r}₁, y^{r}₁ and x^{l}₁, y^{l}ₗ to two points x^{r}₂, y^{r}₂ and x^{l}₂, y^{l}₂, the video sequence is thus processed in such a way that the center point of the pupils remain at the two points x^{r}₁, y^{r}₁ and x^{l}₁, y^{l}₁. The “natural head movement” is thus eliminated and the pumping movement remains in the video sequence, which can then be analyzed with regard to the heart rate.

Inferences about the mental state of a person may also be drawn on the basis of the voice (see, for example, Petri Laukka et al.: In a Nervous Voice: Acoustic Analysis and Perception of Anxiety in Social Phobics' Speech, Journal of Nonverbal Behaviour 32(4): 195-214, December 2008; Owren, M. J., & Bachorowski, J.-A. (2007). Measuring emotion-related vocal acoustics. In J. Coan & J. Allen (Eds.), Handbook of emotion elicitation and assessment (pp. 239-266). New York: Oxford University Press; Scherer, K. R. (2003). Vocal communication of emotion: A review of research paradigms. Speech Communication, 40, 227-256).

In one preferred embodiment, the device according to the invention comprises a (directional) microphone having a connected computer system, using which the voice of a person can be recorded and analyzed. A stress level is determined from the voice pattern. Details are disclosed, for example, in U.S. Pat. No. 7,571,101 B2, WO201552729, WO2008041881, or U.S. Pat. No. 7,321,855.

Illnesses may also be concluded on the basis of mental and/or physical features. This applies above all to features in which the registered values deviate from “normal” values. One example is the “elevated temperature” (fever) already mentioned above, which can indicate an illness.

A very high value of the heart rate or an unusual rhythm of the heart be can also be signs of illnesses.

There are approaches for determining the presence of an illness, for example, Parkinson's disease, from the voice (Sonu R. K. Sharma: Disease Detection Using Analysis of Voice Parameters, TECHNIA—International Journal of Computing Science and Communication Technologies, VOL. 4 NO. 2, January 2012 (ISSN 09743375)).

In addition to a differentiation of the features into physical and mental features, a classification into static or quasistatic and dynamic features could also be performed. Some of the features mentioned typically do not change at all, for example, the sex (static features), others can only change very slowly (quasi-static features), while in contrast others change rapidly (dynamic features). Body size (height), body width, weight, and age are features which typically only change slowly, while heart rate, temperature, and voice are features which can change rapidly.

According to the invention, both static and also quasi-static and also dynamic features of a person are registered. The registration takes place within the same time span. The time span is typically less than one minute, preferably less than 10 seconds, still more preferably less than 5 seconds.

According to the invention, a single device, in which all corresponding means are integrated, is used to register the features of a person.

In addition to the corresponding sensors, this device has means for reading out the sensors and for analyzing the read-out data. For this purpose, one or more computer systems are used. A computer system is a device for electronic data processing by means of programmable computing rules. The device typically has a processing unit, a control unit, a bus unit, a memory, and input and output means according to the von Neumann architecture.

The results of the data analysis are displayed in a visual form. The device according to the invention comprises a first display screen for this purpose.

According to the invention, the raw data determined from the sensors are firstly analyzed to determine features for physical and/or mental states of the analyzed person. Subsequently, visual representations are associated with the determined features and the visual representations are displayed on the first display screen.

Features which may be displayed in the form of numbers (body temperature, heart rate, body size, estimated weight) are preferably displayed as numbers on the first display screen.

Features which may be displayed by means of letters (for example, the sex) are preferably displayed by means of letters (for example, “m” for male and “f” for female). However, it is also conceivable to use symbols for the display of the sex.

Symbols can be used for features which may be displayed only poorly or not at all by means of numbers and/or letters.

For example, the mood derived from the facial analysis and/or voice analysis may be displayed with the aid of an emoticon (for example, “0” for good mood and “0” for bad mood).

Colors can be used to make the displayed items of information more easily comprehensible. For example, a red color could be used for the measured temperature if the temperature is above the normal values (36.0° C.-37.2° C.), while the temperature is displayed in a green color tone if it is within the normal value range.

It is also conceivable that multiple features are summarized in one item of displayed information. For example, if it results from the facial recognition and the heart rate measurement that a person is stressed, a character for a stressed person could be displayed on the first display screen.

The first display screen can be arranged in such a way that it is located directly in the field of view of the analyzed person when the person is analyzed.

It is also conceivable that the first display screen is arranged in such a way that it is located in the field of view of a person (the second person), who interacts or will interact with the analyzed person.

It is also conceivable that the first display screen is arranged so that it can be seen by the first person and by the second person.

The device preferably comprises means, using which the attention of the person to be analyzed can be acquired. The person to be analyzed is thus to position himself in relation to the sensors in such a way that these sensors can better register the corresponding features. It is also conceivable to encourage the person to be analyzed to wait, in order to have the time required to register the features.

In one preferred embodiment, this means for acquiring the attention or for encouraging a longer waiting time is a second display screen.

Images, image sequences, or videos can be played on this second display screen.

It is conceivable to select the items of information displayed on the second display screen in such a way that they are to trigger a reaction in the person to be analyzed. The specific reaction of the person to be analyzed can then be registered by means of suitable sensors, analyzed, and evaluated.

It is conceivable, for example, to display a funny image, which typically causes the person to smile, on the second display screen. If the analyzed person does not display a reaction, this can indicate that they are in a mood in which they do not engage with such jokes.

It is also conceivable to adapt the content of the second display screen to one feature or to multiple features of the analyzed person. The content of the second display screen could, for example, involve personalized advertisement. If it has been recognized by means of the sensors, for example, that the analyzed person is a rather athletic woman in the age between 20 and 30 years, one could thus display products to this person which could interest such persons.

In one preferred embodiment, the device according to the invention is used in a point of sale. The device according to the invention is particularly preferably used in a pharmacy or a comparable business, in which medications and/or health-promoting agents can be purchased.

The device according to the invention is preferably located in the vicinity of the consulting/purchase counter (in the pharmacy, one also refers to the sales counter), so that a person can be analyzed before and/or during a consulting conversation with the pharmacist. Signs or other indications are preferably present which indicate the device. The pharmacy preferably draws attention to the fact that a corresponding device is present and asks the person to be analyzed whether they consent to the registration of features.

The first display screen is preferably arranged in such a way that it can be seen by the pharmacist. A second display screen is preferably provided, which can be seen by the person to be analyzed.

If the person to be analyzed has given their consent to the analysis, features of the person are determined and displayed opposite to the pharmacist, so that the pharmacist can see which needs the person could have in order to be able to consult with them optimally.

The invention will be explained in greater detail hereafter on the basis of a specific example, without wishing to restrict it to the features of the example.

A customer seeks out a pharmacy, in order to consult on a specific, health-related theme. In the running conversation, the pharmacist asks several questions in order to become better acquainted with the need of the customer and be able to assess their situation better. During the conversation, sensors register more extensive parameters of the customer:
a camera registers the face and recognizes sex and age
a second thermal camera measures the body temperature on the forehead of the customer
a third camera measures the heart rate on the basis of the head movements
a directional microphone registers the voice and analyzes the stress level

All registered and analyzed parameters are displayed to the consulting pharmacist, while “personalized advertisement” is displayed to the customer on the basis of their sex and the age. The pharmacist can now orient his consultation on this basis and deal in a more accurate manner with the customer.

All sensors mentioned are installed in a unit. In addition to the sensors mentioned, this unit includes monitors, which display items of information to both the pharmacist and also the customer, and computers (minicomputers), which process the registered items of information (one minicomputer for the facial recognition, one computer for the remaining sensors). In addition, a mobile wireless module offers a remote access to the system in order to, inter alia, play the items of information to be displayed (advertisement) via remote management and/or play back statistical data.

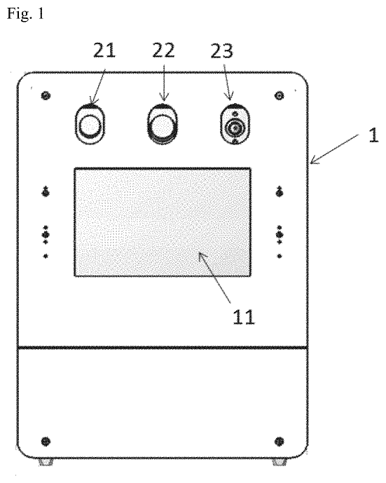

FIG. 1 shows a device (1) according to the invention in a frontal view. The device comprises a display screen (11) for displaying personalized items of information opposite to a person analyzed, and also three sensors (21, 22, 23) for the analysis of the person.

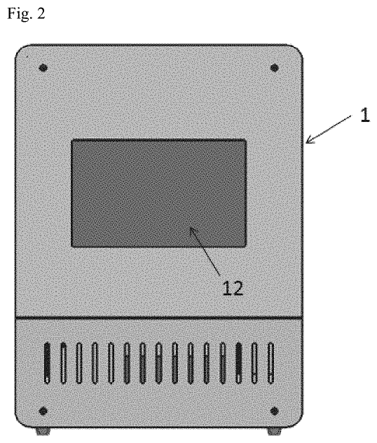

FIG. 2 shows the device (1) according to the invention from FIG. 1 in a rear view. The device comprises a display screen (12) for displaying items of information about the analyzed person opposite to another person.

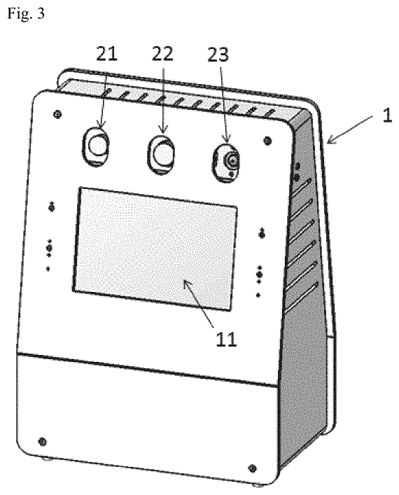

FIG. 3 shows the device (1) according to the invention from FIG. 1 in a perspective illustration from the front and from the side.

1. A device comprising:
one or more sensors for the contactless determination of the following features:
the sex of a first person,
the association of the first person with an age group,
the skin temperature of the first person,
the heart rate of the first person, and
the mood of the first person;
a first display screen;
means for reading out data from the one or more sensors;
means for processing the data read out from the one or more sensors and for associating the read-out and processed data with one or more visual representations of the data; and
means for displaying the one or more visual representations on the first display screen.

2. The device as claimed in claim 1, wherein the one or more sensors comprise one or more image sensors configured to determine the skin temperature, the heart rate, the sex, the association with an age group, and the mood, and wherein the one or more image sensors are connected to a computer system.

3. The device as claimed in claim 2, wherein the determination of the skin temperature, the sex, the association with an age group, and the mood is performed on the basis of the face of the first person.

4. The device as claimed in claim 1, wherein the one or more visual representations are displayed opposite to a second person.

5. The device as claimed in claim 1, comprising a second display screen that is located in the field of vision of the first person during the determination of the features.

6. The device as claimed in claim 5, wherein the second display screen displays items of information in dependence on the determined sex and/or in dependence on the determined association with an age group opposite to the first person.

7. The device as claimed in claim 1, comprising an image sensor connected to a computer system, wherein the computer system is configured to:
analyze an image depicted on the image sensor with respect to a presence of a face; and
if a probability of the presence of the face is greater than a predefined threshold value, analyze the face with respect to the sex and the association with an age group and display the determined items of information about the sex and the association with an age group on the first display screen.

8. The device as claimed in claim 1, comprising a thermal camera configured to determine a temperature of the forehead of the first person, wherein the device is configured to display the determined temperature on the first display screen.

9. The device as claimed in claim 1, comprising: a microphone connected to a computer system, wherein the computer system is configured to register spoken words of the first person using the microphone, carry out a voice analysis to determine a stress level; and display the determined stress level on the first display screen.

10. A method for analyzing a first person, comprising:
recognizing a presence of a first person;
contactlessly determining the following physical and mental features of the first person using one or more sensors:
the sex,
the association with an age group,
the skin temperature,
the heart rate, and
the mood; and
displaying the physical and mental features on a first display screen.

11. The method as claimed in claim 10, wherein items of information are displayed to the first person in dependence on the determined sex and/or in dependence on the association with an age group via a second display screen.

12. The method as claimed in claim 10, wherein the recognition of the presence of the first person, the determination of the sex of the first person, and/or the determination of the association with an age group and/or the heart rate of the first person are performed using an image sensor.

13. The method as claimed in claim 10, wherein the skin temperature is determined using a thermal camera and/or the stress level is determined using a microphone.
